(19) United States
(12) Patent Application Publication
(10) Pub. No.: US 2025/0107692 A1
(43) Pub. Date: Apr. 3, 2025

(54) ENDOSCOPE

(71) Applicant: FUJIFILM Corporation, Tokyo (JP)

(72) Inventor: Takashi HARADA, Kanagawa (JP)

(73) Assignee: FUJIFILM Corporation, Tokyo (JP)

(21) Appl. No.: 18/900,596

(22) Filed: Sep. 27, 2024

(30) Foreign Application Priority Data
Sep. 29, 2023 (JP) 2023-169945

(51) Int. Cl. A61B 1/00; A61B 1/005

(52) U.S. Cl. CPC A61B 1/00098; A61B 1/00101; A61B 1/00137; A61B 1/005

(57) ABSTRACT

Provided is an endoscope capable of improving cleanability of a distal end portion body of an endoscope insertion part. An endoscope includes: a distal end portion body that is provided on a distal end side of an endoscope insertion part; an elevator that is provided on the distal end portion body; a distal end cover that is attachable to and detachable from the distal end portion body; an operation wire that is used to operate the elevator; and a wire insertion passage through which the operation wire is inserted, in which, in a case in which the distal end cover is mounted on the distal end portion body, the distal end cover seals a space portion including at least an outlet of the wire insertion passage.

### CROSS-REFERENCE TO RELATED APPLICATION

The present application claims priority under 35 U.S.C § 119(a) to Japanese Patent Application No. 2023-169945 filed on Sep. 29, 2023, which is hereby expressly incorporated by reference, in its entirety, into the present application.

### BACKGROUND OF THE INVENTION

### 1. Field of the Invention

The present invention relates to an endoscope, and particularly, to an endoscope comprising an elevator on a distal end portion body of an endoscope insertion part.

### 2. Description of the Related Art

In an endoscope, various treatment tools are inserted from a treatment tool inlet port provided at an operation part and are led out from a treatment tool outlet port that is open at a distal end portion of an insertion part, to be used for treatment. For example, in a duodenoscope, treatment tools such as a guide wire or a shaping tube are used. Such a treatment tool needs to change a lead-out direction thereof at the distal end portion of the insertion part in order to treat a desired position in a subject. Therefore, a treatment tool elevation mechanism (elevator) is provided at the distal end portion.

As the treatment tool elevation mechanism, a mechanism (open type) is known in which a wire is attached to the elevator, and the wire is extended to the operation part of the endoscope, and the elevator is swung around a rotary shaft by pushing and pulling the wire with an operation lever provided at the operation part, to change its position between an elevated position and a reclined position.

In addition, a mechanism (closed type) is also known in which the rotary shaft of the elevator is coupled to a housed lever via a partition wall, a wire is attached to the lever, and the elevator is swung around the rotary shaft by pushing and pulling the wire with the operation lever of the operation part, to change its position between the elevated position and the reclined position.

Since the distal end portion provided with such a treatment tool elevation mechanism has a complicated shape and structure, improved cleanability by a cleaning liquid is required.

CN207012171Y discloses an endoscope in which a distal end cover is provided on a distal end portion body of an endoscope insertion part so as to be rotationally movable between a locked position and an unlocked position, and, in a case in which the distal end cover is rotationally moved to the locked position, the distal end cover covers an outlet of a wire insertion passage.

### SUMMARY OF THE INVENTION

However, as with the endoscope in CN207012171Y, simply covering the outlet of the wire insertion passage with the distal end cover may result in contamination of the outlet of the wire insertion passage by a liquid that has entered a gap between the distal end portion body and the distal end cover.

In a case in which the outlet of the wire insertion passage is contaminated, a wire that is in sliding contact with an edge part of the outlet is also contaminated. The wire is composed of a plurality of twisted strands, and, in a case in which the liquid enters a gap between the strands, cleaning the wire itself becomes extremely difficult.

The present invention has been made in view of such circumstances, and an object of the present invention is to provide an endoscope capable of improving cleanability of a distal end portion body of an endoscope insertion part.

According to a first aspect, there is provided an endoscope comprising: a distal end portion body that is provided on a distal end side of an endoscope insertion part; an elevator that is provided on the distal end portion body; a distal end cover that is attachable to and detachable from the distal end portion body; an operation wire that is used to operate the elevator, and a wire insertion passage through which the operation wire is inserted, in which, in a case in which the distal end cover is mounted on the distal end portion body, the distal end cover seals a space portion including at least an outlet of the wire insertion passage.

According to a second aspect, in the endoscope described in the first aspect, the distal end cover includes a first sealing member on a surface facing the space portion.

According to a third aspect, in the endoscope described in the first or second aspect, the distal end portion body includes a lever to which one end of the operation wire is connected and that is used to move the elevator, and a lever housing part that houses the lever, and the distal end cover seals the lever housing part.

According to a fourth aspect, in the endoscope described in the first or second aspect, the distal end portion body includes a partition wall that divides a side on which the elevator is provided and a side on which the wire insertion passage is provided, a through-hole that penetrates between the side on which the elevator is provided and the side on which the wire insertion passage is provided is provided in the partition wall, and the endoscope further comprises a power transmission member that is inserted through the through-hole, with one end connected to the elevator and the other end connected to the operation wire.

According to a fifth aspect, in the endoscope described in the fourth aspect, the power transmission member includes a rotary shaft that is inserted through the through-hole, with one end side connected to the elevator, and a lever that is provided on the other end side of the rotary shaft via a bent portion and to which the operation wire is connected, the distal end portion body includes a lever housing part that houses the lever, and the distal end cover seals the lever housing part.

According to a sixth aspect, in the endoscope described in the fourth or fifth aspect, a second sealing member that seals the through-hole is further provided.

According to a seventh aspect, in the endoscope described in the sixth aspect, the second sealing member is a biocompatible silicone lubricant.

According to an eighth aspect, in the endoscope described in any one of the first to seventh aspects, the distal end cover is mounted to be rotationally movable with respect to the distal end portion body and is rotationally moved between a locked state in which rotation with respect to the distal end portion body is not possible and an unlocked state in which the locked state is released.

According to a ninth aspect, in the endoscope described in the eighth aspect, the distal end portion body includes a hook portion, the distal end cover includes a first hole for allowing insertion and removal of the hook portion and a second hole with which the hook portion is engaged, and a state in which the hook portion is inserted into the first hole is the unlocked state, and a state in which the hook portion is engaged with the second hole is the locked state.

According to a tenth aspect, in the endoscope described in the ninth aspect, the first hole and the second hole are disposed along a rotation direction of the distal end cover.

According to an eleventh aspect, in the endoscope described in the ninth or tenth aspect, the distal end cover includes an opening portion, a stopper portion that is projected from a periphery part of the opening portion toward an inner side of the opening portion is provided, and the first hole and the second hole are disposed on both sides of the stopper portion.

According to a twelfth aspect, in the endoscope described in any one of the first to eleventh aspects, a cap that is mounted on an outer side of the distal end cover is further provided.

According to the present invention, it is possible to improve cleanability of the distal end portion body of the endoscope insertion part.

### BRIEF DESCRIPTION OF THE DRAWINGS

FIG. 1 is a perspective view of an assembly showing a configuration of a distal end portion body of an endoscope insertion part.

FIG. 2 is a perspective view showing a structure of the distal end portion body.

FIG. 3 is a schematic front view of the distal end portion body.

FIG. 4 is an assembly view of the distal end portion body.

FIGS. 5A and 5B are explanatory views of a movement of an elevator moved by an operation of a lever.

FIG. 6 is a perspective view of an appearance of the elevator.

FIG. 7 is an enlarged perspective view showing a configuration of a hook portion.

FIG. 8 is an enlarged perspective view showing a configuration of a distal end cover.

FIG. 9 is a perspective view showing a state before the distal end cover is fixed.

FIG. 10 is a perspective view showing a state after the distal end cover is fixed.

FIG. 11 is an explanatory view of a sealing member provided in the distal end cover.

FIG. 12 is a perspective view of an assembly showing a coupling structure between the elevator and the lever.

FIG. 13 is a cross-sectional view of the coupling structure shown in FIG. 12.

FIG. 14 is a cross-sectional view of a rotary shaft and a through-hole.

FIG. 15 is an explanatory view of a void portion defined by the through-hole and the rotary shaft.

### DESCRIPTION OF THE PREFERRED EMBODIMENTS

Hereinafter, an endoscope according to an embodiment of the present invention will be described.

### Embodiment

In the embodiment, a side-viewing endoscope used as a duodenoscope is exemplified. The endoscope comprises an operation part that is operated by an operator and an endoscope insertion part (hereinafter, referred to as an “insertion part”) that is connected to the operation part and that is inserted into a subject.

The insertion part is inserted into the subject through an oral cavity and is further inserted into a duodenum from an esophagus through a stomach. As a result, a predetermined examination or treatment, such as medical therapy, using a treatment tool inserted into the insertion part is performed on the duodenum.

Examples of the treatment tool include biopsy forceps whose distal end portion includes a cup that allows collection of a biological tissue, a knife for endoscopic sphincterotomy (EST), or a contrast tube.

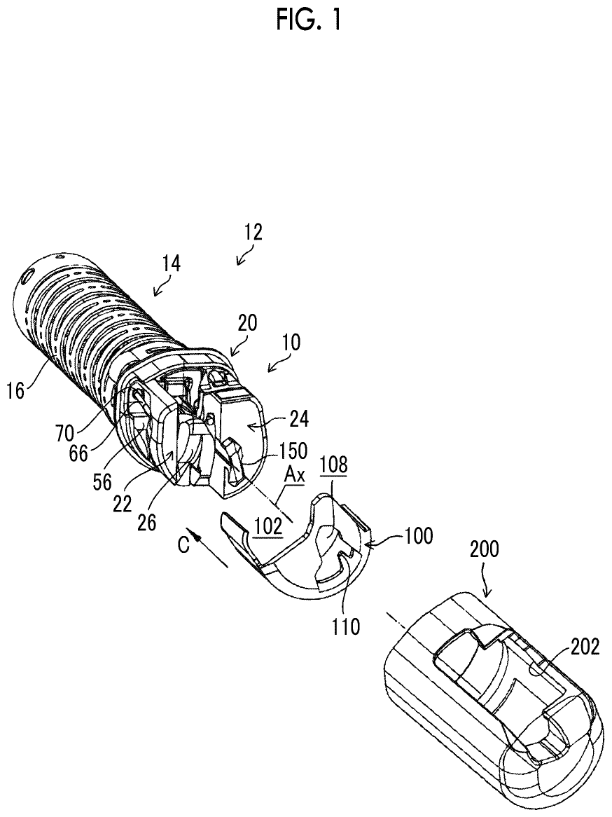

FIG. 1 is a perspective view of an assembly showing a configuration of a distal end portion 10 including a distal end portion body 20 according to the embodiment. The distal end portion body 20 is provided on a distal end side of an insertion part 12. The distal end portion body 20 is an example of a distal end portion body of the embodiment of the present invention.

As shown in FIG. 1, the insertion part 12 has a major axis Ax extending from a proximal end side connected to the operation part to the distal end side and comprises a soft portion, a bending portion 14, and the distal end portion 10 in this order from the proximal end side to the distal end side.

The bending portion 14 includes a cylindrical member 16 that is configured to bend by providing notch portions in a circumferential direction, and has a configuration in which an outer periphery of the cylindrical member 16 is covered with a mesh-like body woven with a metal wire and is further covered with a rubber outer sheath. In addition, a plurality of wires extend from an angle knob of the operation part to the bending portion 14, and distal end portions of these wires are fixed to the cylindrical member 16 constituting the bending portion 14. As a result, the bending portion 14 bends up, down, left, and right in response to an operation of the angle knob.

The distal end portion 10 comprises the distal end portion body 20, a distal end cover 100 that is attachable to and detachable from the distal end portion body 20, and a cap 200 that is mounted on an outer side of the distal end cover 100.

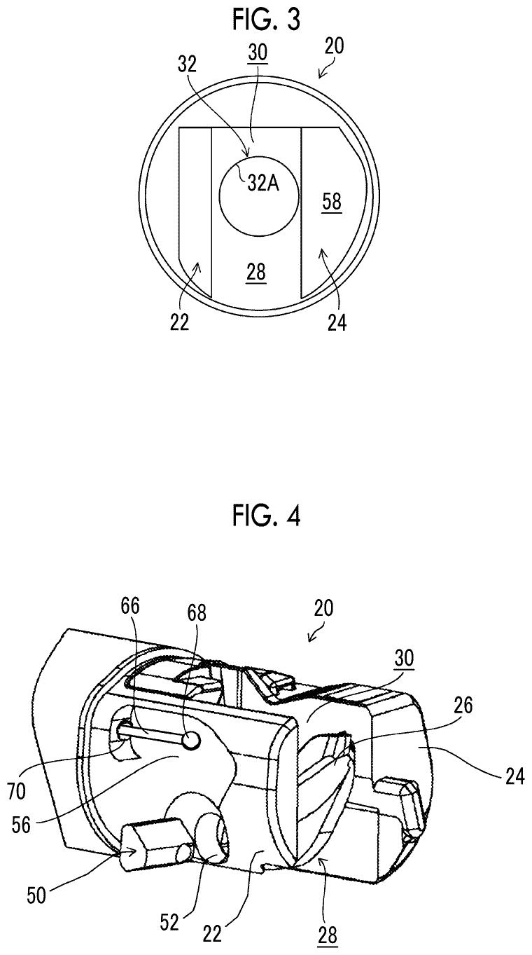

The distal end cover 100 includes an open part 102 through which an opening 30 on an upper surface side of an elevator housing chamber 28 (refer to FIG. 3) to be described below is open in a state in which the distal end cover 100 is mounted on the distal end portion body 20. The distal end cover 100 is made of a metal material having corrosion resistance. The distal end cover 100 will be described below. The distal end cover 100 is an example of a distal end cover of the embodiment of the present invention.

Additionally, the cap 200 includes an opening window 202 through which the above- described opening 30 is open in a state in which the cap 200 is mounted on the distal end portion body 20. The cap 200 includes an engaging portion that is provided on the proximal end side thereof and that is engaged with a groove formed in the distal end portion body 20, and is attachably and detachably mounted on the distal end portion body 20. The cap 200 consists of an elastic material or a flexible material, for example, silicone rubber or polyethylene. The cap 200 is an example of a cap of the embodiment of the present invention.

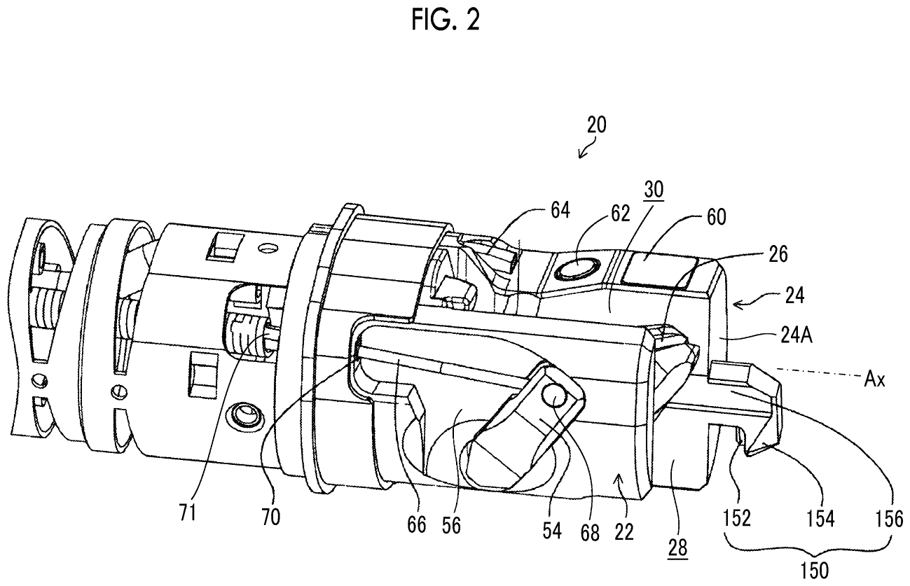

FIG. 2 is a perspective view of the distal end portion body 20. FIG. 3 is a schematic front view of the distal end portion body 20.

As shown in FIGS. 2 and 3, the distal end portion body 20 includes a pair of first and second partition walls 22 and 24 that are a part of the distal end portion body 20 and that face each other. The elevator housing chamber 28, which is a slit-shaped space for housing an elevator 26 (not shown in FIG. 3), is formed between the first partition wall 22 and the second partition wall 24. An opening of the elevator housing chamber 28 on the upper surface side in FIG. 2 is formed as the above-described opening 30. The distal end portion body 20 is made of a metal material having corrosion resistance.

In addition, a treatment tool insertion channel 32 communicates with the elevator housing chamber 28 of the distal end portion body 20. The treatment tool insertion channel 32 is inserted into the insertion part 12 and connected to a treatment tool inlet port of the operation part. As a result, the treatment tool introduced from the treatment tool inlet port is led out to the elevator housing chamber 28 through the treatment tool insertion channel 32.

The elevator 26 is provided on the distal end portion body 20 and is housed in the elevator housing chamber 28. The elevator 26 changes a direction of the treatment tool led out to the elevator housing chamber 28 from a treatment tool outlet port 32A, which is a distal end opening of the treatment tool insertion channel 32, causing the treatment tool to protrude through the opening 30. The elevator 26 is swingably attached to a through-hole 52 of the first partition wall 22 via a rotary shaft 50 (refer to FIG. 4), which will be described below, and can control a lead-out direction of the treatment tool in a case in which the treatment tool is led out from the treatment tool outlet port 32A. The elevator 26 is an example of an elevator of the embodiment of the present invention.

A recessed lever housing chamber 56 that houses a lever 54 is formed on a surface of the first partition wall 22 on a side opposite to a facing surface side facing the elevator housing chamber 28. The lever 54 and the lever housing chamber 56 are examples of a lever and a lever housing part of the embodiment of the present invention.

A housing chamber 58 that houses an optical system and the like is provided in the second partition wall 24. An irradiation window 60, an observation window 62, and an air and water supply nozzle 64 directed toward the observation window 62 are provided at an upper part of the housing chamber 58. The air and water supply nozzle 64 is connected to an air and water supply device via an air and water supply tube inserted into the insertion part 12. By operating an air and water supply button of the operation part, air or water is jetted from the air and water supply nozzle 64 toward the observation window 62, and the observation window 62 is cleaned.

In addition, an illumination unit and an imaging unit are housed in the second partition wall 24. The illumination unit comprises an illumination lens installed on an inner side of the irradiation window 60 and a light guide whose distal end is disposed to face the illumination lens. The light guide is inserted into the insertion part 12, and a proximal end portion thereof is connected to a light source device. As a result, irradiation light from the light source device is transmitted via the light guide and is emitted from the irradiation window 60.

The imaging unit comprises an imaging optical system disposed on an inner side of the observation window 62, and a complementary metal-oxide-semiconductor (CMOS) type imaging element or a charge-coupled device (CCD) type imaging element. The imaging element is connected to an image processing device via a signal cable inserted into the insertion part 12. An imaging signal of a subject image obtained by the imaging using the imaging unit is input to the image processing device via the signal cable, and the subject image is displayed on a monitor connected to the image processing device.

FIG. 4 is a perspective view of an assembly of the distal end portion body 20. In FIG. 4, the lever 54 is not shown, and the rotary shaft 50 provided on the lever 54 is shown.

As shown in FIG. 4, the through-hole 52 that penetrates the first partition wall 22 and that communicates with the elevator housing chamber 28 is provided in a bottom surface of the recessed lever housing chamber 56. The rotary shaft 50 that couples the elevator 26 and the lever 54 to each other is inserted through the through-hole 52. That is, the first partition wall 22 is provided with the through-hole 52 through which the rotary shaft 50 is inserted. Since the lever 54 in the lever housing chamber 56 swings about the rotary shaft 50, the lever housing chamber 56 is formed in a substantially fan shape centered on the rotary shaft 50. The rotary shaft 50 and the through-hole 52 are examples of a rotary shaft and a through-hole of the embodiment of the present invention.

As will be described in a detailed configuration below, one end side of the lever 54 is coupled to the elevator 26 via the rotary shaft 50 and the other end side thereof is connected to an operation wire 66. The lever 54 swings about the rotary shaft 50 integrally with the elevator 26 with operation of the operation wire 66.

As shown in FIG. 2, the operation wire 66 includes a connection portion 68 that is provided on a distal end side of the operation wire 66 and that is connected to the lever 54 in the lever housing chamber 56. That is, one end of the operation wire 66 is connected to the lever 54 for moving the elevator 26. In addition, a proximal end side of the operation wire 66 is coupled to an elevator operating mechanism in the operation part by being inserted through a wire insertion passage 71 from an outlet 70 that is open at a wall surface of the lever housing chamber 56. The operation wire 66 and the wire insertion passage 71 are examples of an operation wire and a wire insertion passage of the embodiment of the present invention.

With the distal end portion body 20 configured in this way, the first partition wall 22 that divides a side on which the elevator 26 is provided (elevator housing chamber 28) and a side on which the wire insertion passage 71 is provided (lever housing chamber 56) is provided. In addition, the first partition wall 22 is provided with the through-hole 52 that communicates between the side on which the elevator 26 is provided and the side on which the wire insertion passage is provided. The first partition wall 22 is an example of a partition wall of the embodiment of the present invention.

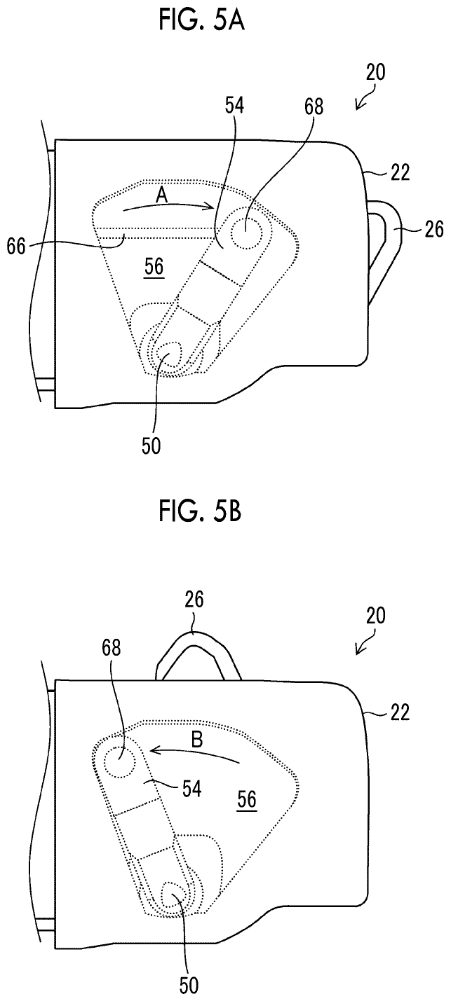

FIGS. 5A and 5B are explanatory views illustrating movements of the elevator 26 moved by an operation of the lever 54.

As shown in FIG. 5A, in a case in which the operation wire 66 is pushed by an operation of an operation lever, the lever 54 swings in an A direction about the rotary shaft 50, and the elevator 26 moves from an elevated position toward a reclined position. On the other hand, as shown in FIG. 5B, in a case in which the operation wire 66 is pulled by the operation of the operation lever, the lever 54 swings in a B direction (a direction opposite to the A direction) about the rotary shaft 50, and the elevator 26 moves from the reclined position to the elevated position. In this way, the operation wire 66 can rotationally move the rotary shaft 50 via the lever 54 through the operation of the operation lever. Consequently, a position of the elevator 26 is changed between the elevated position and the reclined position. The lever 54 and the rotary shaft 50 may be provided integrally with each other or may be separately provided so as to be attachable to and detachable from each other. The lever 54 and the rotary shaft 50 are coupled to each other via a bent portion and are an example of a power transmission member of the embodiment of the present invention.

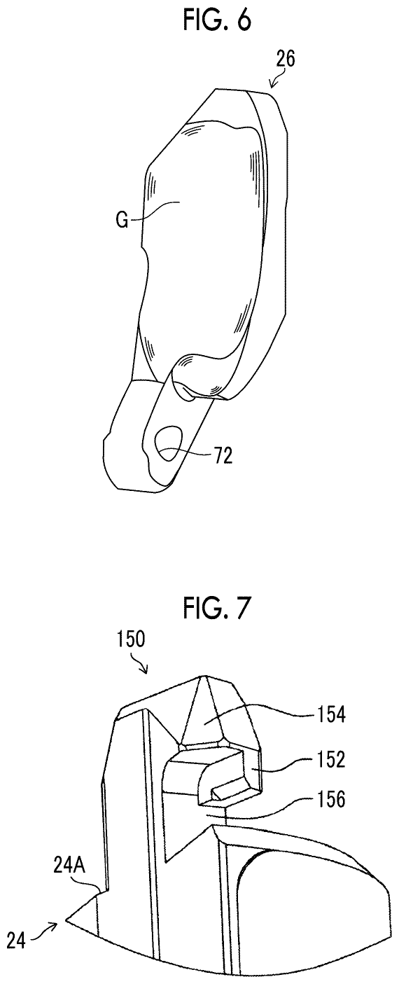

FIG. 6 is a perspective view of an appearance of the elevator 26 housed in the elevator housing chamber 28.

As shown in FIG. 6, the elevator 26 includes a circular arc-shaped guide surface G. The guide surface G faces the treatment tool outlet port 32A of the treatment tool insertion channel 32 in the elevator housing chamber 28 and guides, toward the opening 30, the treatment tool led out from the treatment tool outlet port 32A to the elevator housing chamber 28.

A proximal end portion of the elevator 26 is provided with a through-hole 72 into which the rotary shaft 50 is inserted. The rotary shaft 50 is inserted into the through-hole 72, whereby the elevator 26 is coupled to the lever 54 via the rotary shaft 50.

### Distal End Cover

Next, returning to FIG. 1, the distal end cover 100 will be described. The distal end cover 100 is a member that seals a space portion including at least the outlet 70 of the wire insertion passage 71 in a case in which the distal end cover 100 is mounted on the distal end portion body 20. The distal end cover 100 is mounted on the distal end portion body 20 by using a hook portion 150 provided on the distal end portion body 20. First, the hook portion 150 will be described.

### Hook Portion

As shown in FIG. 2, the hook portion 150 is provided on a distal end surface 24A of the second partition wall 24 and is projected from the distal end surface 24A along a direction of the major axis Ax (forward direction). The hook portion 150 includes an engaging part 152, a contact part 154, and a coupling part 156 and is configured in an L-like shape as a whole.

FIG. 7 is an enlarged perspective view showing a configuration of the hook portion 150. As shown in FIG. 7, the coupling part 156 of the hook portion 150 protrudes forward from the distal end surface 24A of the second partition wall 24. The contact part 154 protrudes from a distal end of the coupling part 156 toward a lower side of FIG. 2 so as to be parallel to the distal end surface 24A of the second partition wall 24. The engaging part 152 protrudes from a surface side of the contact part 154, which faces the distal end surface 24A, toward the distal end surface 24A. The above is the configuration of the hook portion 150. The hook portion 150 is an example of a hook portion of the embodiment of the present invention.

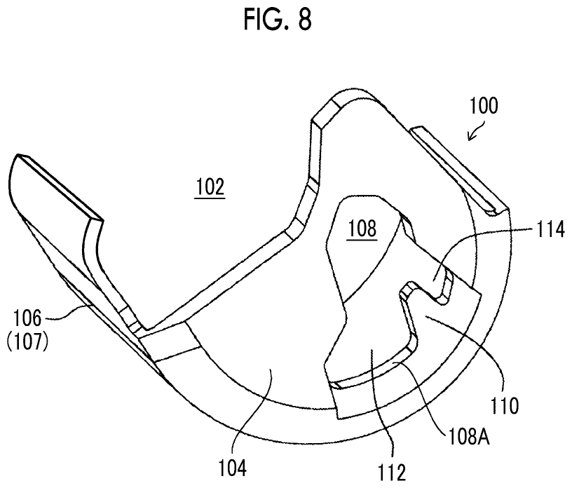

FIG. 8 is an enlarged perspective view showing a configuration of the distal end cover 100. As shown in FIG. 8, the distal end cover 100 includes a fan-shaped distal end wall 104 and a semi-cylindrical outer peripheral wall 106 provided at a peripheral part of the distal end wall 104. In a case in which the distal end cover 100 is mounted on the distal end portion body 20, the elevator 26 and the treatment tool are led out from the open part 102 provided in the outer peripheral wall 106.

The distal end wall 104 includes a substantially fan-shaped opening portion 108. A periphery part 108A of the opening portion 108 includes a stopper portion 110. The stopper portion 110 is projected from the periphery part 108A toward an inner side of the opening portion 108. A first hole 112 and a second hole 114 are disposed on both sides of the stopper portion 110. The first hole 112 and the second hole 114 are disposed along a rotation direction of the distal end cover 100, which will be described below. The opening portion 108, the stopper portion 110, the first hole 112, and the second hole 114 are examples of an opening portion, a stopper portion, a first hole, and a second hole of the embodiment of the present invention.

A size of the first hole 112 is configured to be larger than a size of the hook portion 150 (engaging part 152). A size of the second hole 114 is configured to be smaller than the size of the hook portion 150 (engaging part 152). This allows the hook portion 150 to protrude forward from the first hole 112, and by rotating the distal end cover 100, the hook portion 150 (engaging part 152) can be engaged with the second hole 114.

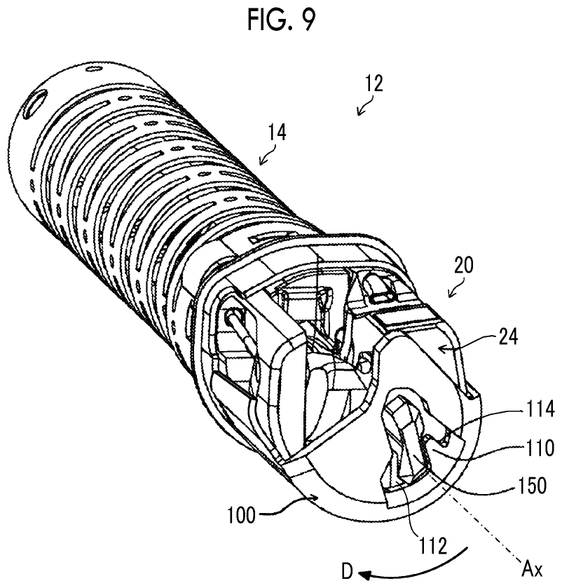

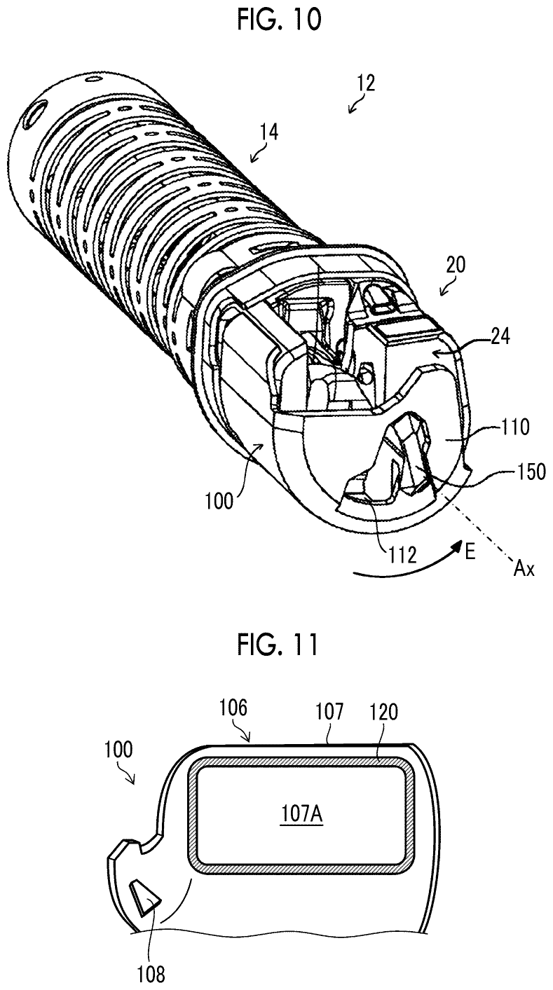

Here, a fixing method of the distal end cover 100 with respect to the distal end portion body 20 will be described with reference to FIGS. 9 and 10. FIG. 9 is a perspective view showing a state before fixing, and FIG. 10 is a perspective view showing a state after fixing.

As shown in FIG. 9, the distal end cover 100 is mounted to be rotationally movable with respect to the distal end portion body 20. The distal end cover 100 is rotationally moved with respect to the distal end portion body 20 between a locked state in which rotation with respect to the distal end portion body 20 is not possible (refer to FIG. 10) and an unlocked state in which the locked state is released (refer to FIG. 9). The rotational movement of the distal end cover 100 is performed with the major axis Ax as a rotational movement axis.

Specifically, the distal end cover 100 is mounted on the distal end portion body 20 from a direction of an arrow C (refer to FIG. 1). In this case, the engaging part 152 of the hook portion 150 is inserted into the first hole 112 and protrudes forward from the distal end wall 104, resulting in the unlocked state shown in FIG. 9. That is, the state in which the hook portion 150 is inserted into the first hole 112 is the unlocked state, and the rotational movement of the distal end cover 100 with respect to the distal end portion body 20 is allowed.

Next, in a case in which the distal end cover 100 is rotationally moved in a direction of an arrow D in FIG. 9, the stopper portion 110 enters a gap between the engaging part 152 and the distal end surface 24A, and the engaging part 152 is engaged with the second hole 114 in this state. Consequently, the distal end cover 100 is fixed to the distal end portion body 20, resulting in the locked state. That is, the state in which the hook portion 150 is engaged with the second hole 114 is the locked state, and an unintended rotational movement of the distal end cover 100 with respect to the distal end portion body 20 is restricted. The cap 200 (refer to FIG. 1) is mounted on the outer side of the distal end cover 100.

In a case in which the distal end cover 100 is removed from the distal end portion body 20, the distal end cover 100 in the locked state shown in FIG. 10 is rotationally moved in a direction of an arrow E (opposite to the direction of the arrow D) to change the locked state to the unlocked state shown in FIG. 9. As a result, the distal end cover 100 can be removed from the distal end portion body 20.

Here, as shown in FIG. 10, in a case in which the liquid has entered the lever housing chamber 56 through a gap between the distal end portion body 20 and the distal end cover 100 in a state in which the distal end cover 100 is mounted on (fixed to) the distal end portion body 20, the lever 54, the outlet 70 of the wire insertion passage 71, and the like may be contaminated by the liquid. In a case in which the outlet 70 of the wire insertion passage 71 is contaminated, the operation wire 66 that is in contact with the edge part of the outlet 70 is also contaminated. The operation wire 66 is composed of a plurality of twisted strands, and, in a case in which the liquid enters a gap between the strands, cleaning the operation wire 66 itself becomes extremely difficult.

In that respect, the distal end portion body 20 of the embodiment has the following configuration (sealing structure) in order to solve such a problem of the cleanability and to improve the cleanability of the distal end portion body 20.

### Sealing Structure of Space Portion Including Outlet of Wire Insertion Passage

With the distal end portion body 20 of the embodiment, in a case in which the distal end cover 100 is mounted on the distal end portion body 20 (refer to FIG. 10), the distal end cover 100 seals the space portion including at least the outlet 70 of the wire insertion passage 71. Sealing refers to water-tightly shielding the space portion including the outlet 70 of the wire insertion passage 71 from an external space of the space portion. On the other hand, covering merely refers to covering the space portion of the outlet 70 of the wire insertion passage 71, without providing a function of water-tightly shielding the space portion, such as sealing.

With the distal end portion body 20 of the embodiment, in a case in which the distal end cover 100 is mounted on the distal end portion body 20, the space portion including at least the outlet 70 of the wire insertion passage 71 is sealed by the distal end cover 100, so that the contamination of the outlet 70 of the wire insertion passage 71 by the liquid can be prevented even in a case in which the liquid has entered the gap between the distal end portion body 20 and the distal end cover 100. Consequently, it is possible to improve the cleanability of the distal end portion body 20. In addition, in a case in which the lever housing chamber 56 is also sealed by the distal end cover 100, the contamination of the lever 54 by the liquid can be prevented, so that it is possible to improve the cleanability of the distal end portion body 20.

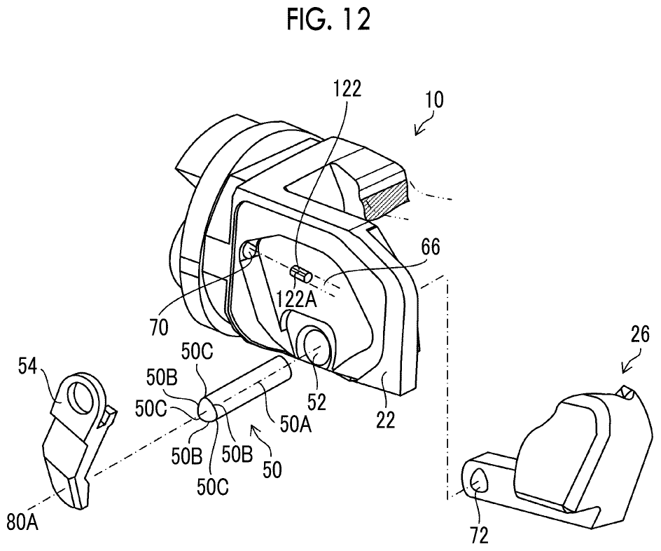

FIG. 11 is an explanatory view showing an example of the sealing structure for sealing the above-described space portion with the distal end cover 100.

As shown in FIG. 11, the distal end cover 100 includes a sealing member 120 on an inner surface 107A of an outer wall 107 of the outer peripheral wall 106, which faces the lever housing chamber 56. The sealing member 120 is configured in a frame shape on the inner surface 107A, and, in a case in which the distal end cover 100 is mounted on the distal end portion body 20 (refer to FIG. 10), the sealing member 120 is in close contact with the distal end portion body 20 (first partition wall 22) to surround the outlet 70 of the wire insertion passage 71 formed in the first partition wall 22 and the lever housing chamber 56.

In this case, the space portion including the outlet 70 of the wire insertion passage 71 and a space portion including the lever housing chamber 56 are space portions water-tightly shielded from the external space by the distal end portion body 20, the distal end cover 100, and the sealing member 120. Consequently, the space portion including the outlet 70 of the wire insertion passage 71 is sealed. In addition, the distal end cover 100 also seals the lever housing chamber 56.

As the sealing member 120, a frame-shaped elastic member (for example, made of rubber) can be exemplified. In this case, the sealing member 120 can be configured with a frame-shaped elastic member disposed on the inner surface 107A. The inner surface 107A of the outer wall 107 is an example of a surface facing a space portion of the embodiment of the present invention, and the sealing member 120 is an example of a first sealing member.

By providing the distal end cover 100 with such a sealing member 120, the space portion including at least the outlet 70 of the wire insertion passage 71 can be reliably sealed, so that the contamination of the outlet 70 by the liquid can be prevented. Consequently, it is possible to improve the cleanability of the distal end portion body 20. In addition, since the lever housing chamber 56 is also sealed by the sealing member 120, it is possible to prevent the contamination of the lever 54 by the liquid.

As a contamination prevention measure of the outlet 70, by filling the outlet 70 with a lubricant, the outlet 70 may be sealed. Examples of the lubricant in this case include a biocompatible silicone lubricant. In addition, as another contamination prevention measure, by fitting a tubular sealing member 122 (refer to FIG. 12) into the outlet 70, the contamination of the outlet 70 may be prevented. The sealing member 122 includes a slit 122A formed along an axial direction, and the operation wire 66 can be inserted through an inner side of the sealing member 122 via the slit 122A.

Meanwhile, the distal end portion body 20 is a member that is exposed to a liquid such as a body fluid during use of the endoscope. Therefore, the liquid may flow from the elevator housing chamber 28 to the through-hole 52 of the first partition wall 22 and may enter the lever housing chamber 56. In this case, since the rotary shaft 50, the through-hole 52, the lever 54, and the like may be contaminated by the liquid, these components should be cleaned. However, it is difficult to completely clean these components because these components are small and have complicated shapes.

In that respect, the distal end portion body 20 of the embodiment has the following configuration in order to solve the above-described problem of the cleanability and to improve the cleanability of the distal end portion body 20.

### Sealing Structure of Through-Hole

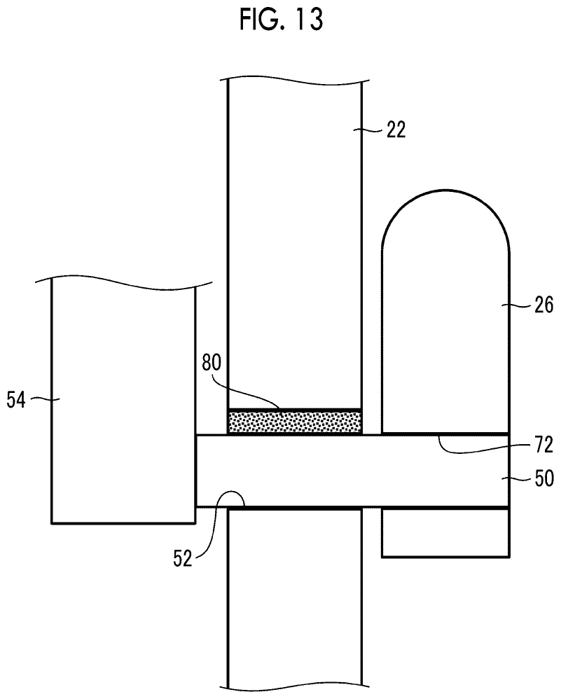

FIG. 12 is a perspective view of an assembly showing the coupling structure between the elevator 26 and the lever 54 for sealing the through-hole 52. FIG. 13 is a cross-sectional view of the coupling structure shown in FIG. 12.

### Rotary Shaft

First, the rotary shaft 50 will be described. The rotary shaft 50 is formed with a side part 50B in which a peripheral surface parallel to a direction of an axis 50A of the rotary shaft 50 forms a planar shape, and an arc part 50C in which the peripheral surface forms a curved shape. Specifically, the rotary shaft 50 has a substantially triangular prism shape in which three side parts 50B and three arc parts 50C are alternately formed in a peripheral direction of the rotary shaft 50 and the side parts 50B and the arc parts 50C are extended along the direction of the axis 50A.

In a case in which such a rotary shaft 50 is inserted through the through-hole 52 in order to couple the elevator 26 and the lever 54 to each other, as shown in the cross-sectional view of the rotary shaft 50 and the through-hole 52 shown in FIG. 14, the arc part 50C of the rotary shaft 50 is in sliding contact with an inner surface 52A of the through-hole 52, which is a round hole, but the side part 50B of the rotary shaft 50 is not in contact with the inner surface 52A. Therefore, a void portion 53 is formed between the side part 50B and the inner surface 52A. In the present example, the void portion 53 is formed at three locations in the peripheral direction of the rotary shaft 50.

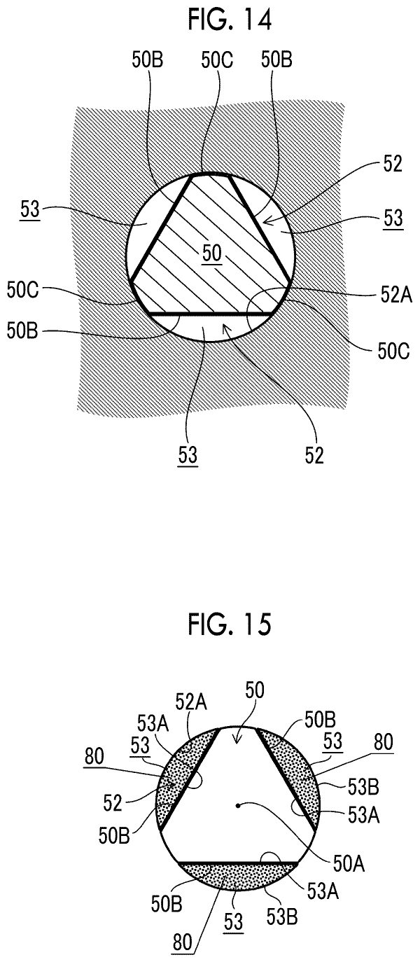

FIG. 15 is an explanatory view of the void portion 53 provided between the side part 50B of the rotary shaft 50 and the inner surface 52A of the through-hole 52.

As shown in FIG. 15, the void portion 53 is a space portion defined by the side part 50B and the inner surface 52A and is formed in a substantially semilunar shape having a void-side side part 53A along the side part 50B and a void-side arc part 53B along the inner surface 52A.

Since the void portion 53 formed in this way serves as a flow passage of the fluid from the elevator housing chamber 28 toward the lever housing chamber 56, it is necessary to close the void portion 53 and seal the through-hole 52 in order to improve the cleanability of the distal end portion body 20. In that respect, the distal end portion body 20 of the embodiment comprises a sealing member 80 for sealing the through-hole 52 in the coupling structure between the elevator 26 and the lever 54. Hereinafter, the sealing member 80 will be described.

### Sealing Member

In the present example, a lubricant is used as the sealing member 80. As shown in FIG. 15, the three void portions 53 are filled with the sealing member 80, which is the lubricant. Consequently, since the through-hole 52 is sealed by the sealing member 80, a flow of the fluid from the elevator housing chamber 28 toward the lever housing chamber 56 can be blocked by the sealing member 80. Examples of the lubricant include a biocompatible silicone lubricant. The sealing member 80 is an example of a second sealing member of the embodiment of the present invention.

As described above, with the distal end portion body 20 of the embodiment, the sealing member 80 that seals the through-hole 52 is provided, so that it is possible to prevent the liquid such as the body fluid from entering the lever housing chamber 56 from the through-hole 52. As a result, it is possible to improve the cleanability of the distal end portion body 20.

### Modification Example

Hereinafter, some modification examples will be described.

### First Modification Example

In the embodiment, the frame-shaped elastic member disposed on the inner surface 107A has been exemplified as the sealing member 120, but the present invention is not limited to this. For example, as another sealing member, a frame-shaped protrusion portion may be integrally formed on the inner surface 107A, and the protrusion portion may be brought into close contact with the distal end portion body 20 to seal the above-described space portion. In addition, the biocompatible silicone lubricant may be applied to the inner surface 107A in a frame shape, and the biocompatible silicone lubricant may be brought into close contact with the distal end portion body 20 to seal the above-described space portion.

In each of the above-described embodiments, the side-viewing endoscope has been described as an example, but the present invention can be applied to various endoscopes such as an ultrasonic endoscope and a direct-viewing endoscope, which comprise an elevator that is provided at the distal end portion of the insertion part and that changes the lead-out direction of the treatment tool.

Although the endoscope according to the embodiment has been described above, the present invention may be subjected to some improvements or modifications without departing from the gist of the present invention.

### EXPLANATION OF REFERENCES

10: distal end portion
12: insertion part
14: bending portion
16: cylindrical member
20: distal end portion body
22: first partition wall
24: second partition wall
24A: distal end surface
26: elevator
28: elevator housing chamber
30: opening
32: treatment tool insertion channel
32A: treatment tool outlet port
50: rotary shaft
50A: axis
50B: side part
50C: arc part
52: through-hole
52A: inner surface
53: void portion
53A: void-side side part
53B: void-side arc part
54: lever
56: lever housing chamber
58: housing chamber
60: irradiation window
62: observation window
64: air and water supply nozzle
66: operation wire
68: connection portion
70: outlet
71: wire insertion passage
72: through-hole
72B: hole-side side part
72C: hole-side arc part
80: sealing member
100: distal end cover
102: open part
104: distal end wall
106: outer peripheral wall
107: outer wall
107A: inner surface
108: opening portion
108A: periphery part
110: stopper portion
112: first hole
114: second hole
122: sealing member
122A: slit
150: hook portion
152: engaging part
154: contact part
156: coupling part
200: cap
202: opening window
Ax: major axis
G: guide surface

What is claimed is:

1. An endoscope comprising:
a distal end portion body that is provided on a distal end side of an endoscope insertion part;
an elevator that is provided on the distal end portion body;
a distal end cover that is attachable to and detachable from the distal end portion body;
an operation wire that is used to operate the elevator; and
a wire insertion passage through which the operation wire is inserted,
wherein, in a case in which the distal end cover is mounted on the distal end portion body, the distal end cover seals a space portion including at least an outlet of the wire insertion passage.

2. The endoscope according to claim 1,
wherein the distal end cover includes a first sealing member on a surface facing the space portion.

3. The endoscope according to claim 1,
wherein the distal end portion body includes
a lever to which one end of the operation wire is connected and that is used to move the elevator, and
a lever housing part that houses the lever, and the distal end cover seals the lever housing part.

4. The endoscope according to claim 1,
wherein the distal end portion body includes a partition wall that divides a side on which the elevator is provided and a side on which the wire insertion passage is provided,
a through-hole that penetrates between the side on which the elevator is provided and the side on which the wire insertion passage is provided is provided in the partition wall, and
the endoscope further comprises a power transmission member that is inserted through the through-hole, with one end connected to the elevator and the other end connected to the operation wire.

5. The endoscope according to claim 4,
wherein the power transmission member includes
a rotary shaft that is inserted through the through-hole, with one end side connected to the elevator, and
a lever that is provided on the other end side of the rotary shaft via a bent portion and to which the operation wire is connected,
the distal end portion body includes a lever housing part that houses the lever, and
the distal end cover seals the lever housing part.

6. The endoscope according to claim 4, further comprising:
a second sealing member that seals the through-hole.

7. The endoscope according to claim 6,
wherein the second sealing member is a biocompatible silicone lubricant.

8. The endoscope according to claim 1,
wherein the distal end cover is mounted to be rotationally movable with respect to the distal end portion body and is rotationally moved between a locked state in which rotation with respect to the distal end portion body is not possible and an unlocked state in which the locked state is released.

9. The endoscope according to claim 8,
wherein the distal end portion body includes a hook portion,
the distal end cover includes a first hole for allowing insertion and removal of the hook portion and a second hole with which the hook portion is engaged, and
a state in which the hook portion is inserted into the first hole is the unlocked state, and a state in which the hook portion is engaged with the second hole is the locked state.

10. The endoscope according to claim 9,
wherein the first hole and the second hole are disposed along a rotation direction of the distal end cover.

11. The endoscope according to claim 10,
wherein the distal end cover includes an opening portion,
a stopper portion that is projected from a periphery part of the opening portion toward an inner side of the opening portion is provided, and
the first hole and the second hole are disposed on both sides of the stopper portion.

12. The endoscope according to claim 11, further comprising:
a cap that is mounted on an outer side of the distal end cover.
